(19) United States
(12) Patent Application Publication
(10) Pub. No.: US 2010/0186780 A1
(43) Pub. Date: Jul. 29, 2010

(54) DENTAL APPLICANCE CLEANING APPARATUS

(76) Inventors: Michael J. Larocca, Parsippany, NJ (US); Stanley J. Lech, Parsippany, NJ (US); Naresh I. Mehta, Parsippany, NJ (US); Stephen B. McAuliffe, Parsippany, NJ (US); Brendyn Murray Rodgers, Victoria (AU); Kon Euan Gerard Wong, Victoria (AU); Andrew Zipsin, Victoria (AU)

Correspondence Address: GlaxoSmithKline;GLOBAL PATENTS -US, UW2220, P. O. BOX 1539, KING OF PRUSSIA, PA, 19406-0939, US

(21) Appl. No.: 12/664,947

(22) PCT Filed: Jun. 18, 2008
(86) PCT No.: PCT/US08/67304

§ 371 Date: Dec. 16, 2009

Related U.S. Application Data
(60) Provisional application No. 60/936,057, filed on Jun. 18, 2007.

(51) Int. Cl. B08B 3/00

(52) U.S. Cl. 134/105

(57) ABSTRACT

The present disclosure is an apparatus that includes a housing having a lid pivotally connected to the housing. The lid has an open position and a closed position and a wall with a heating element connected thereto. A removable container can fit inside the housing, and can be divided into a first compartment and a second compartment when the lid is in its closed position. The heating element emits heat when it is activated. The wall is operatively arranged to divide the container into the first compartment and the second compartment when the lid is in the closed position such that the contents of the first compartment are prevented from entering the second compartment.

### BACKGROUND OF THE INVENTION

1. Field of the Invention

The present disclosure relates to an apparatus for cleaning dental appliances. More particularly, the present disclosure relates to an apparatus for cleaning dental appliances that heats a solution to clean dental appliances and provides a component that cools the heated solution and/or dental appliance.

2. Description of Related Art

Mainstream methods of cleaning dental appliances, such as dentures, bridges, orthodontic retainers and appliances, protective mouthguards, and nightguards to prevent bruxism and/or temporomandibular joint (TMJ) syndrome, typically use brushing or soaking to cleanse the appliance. However, certain dental appliances have surfaces that are porous or have crevices that are not reached by mere brushing or soaking, or a combination of those techniques.

Pores and crevices in the surface of dental appliances may entrap matter that promotes the development of bacterial growth. The proliferation of bacteria in pores and crevices of dental appliances has proven to a major concern for wearers of dental appliances. The bacterial colonies tend to produce unpleasant odors that effect the quality of life of the wearer of dental appliances. Traditional cleansing techniques and devices fail to penetrate the pores and provide a deep clean of the pores and crevices. Thus, traditional cleansing techniques and devices do little if anything to eliminate or hinder the growth of bacterial colonies in these pores and crevices.

Heat has been implemented to improve the effectiveness of traditional cleaning techniques. However, there are risks and problems associated with cleaning techniques that use heat. Foremost of the risks and problems associated with using heat in the cleaning process is the potential for injury to the user, and possible damage to the dental appliances. Dental appliances that are exposed to heat during the cleaning process can injure the user if not cooled properly. Another problem associated with deep cleaning techniques that use heat is that cooling periods can be prolonged and injury can occur if the dental appliance is not cooled sufficiently.

Accordingly, there is a need for a cleaning apparatus and method that overcome, alleviate, and/or mitigate one or more of the aforementioned and other deleterious effects of prior art cleaning devices and methods.

### BRIEF SUMMARY OF THE INVENTION

The present disclosure provides a device for cleaning dental appliances that deep cleans dental appliances to remove deposits and debris that could promote bacterial growth.

The present disclosure also provides a method and device for cleaning dental appliances that has a mechanism to dispense a substance to clean dental appliances.

The present disclosure further provides a method and device for cleaning dental appliances that has a container with one compartment that functions as a cleaning compartment that can be heated, and another compartment that cools the cleaning compartment and/or the dental appliance contained inside the cleaning compartment.

These and other advantages and benefits of the present disclosure are provided by an apparatus including a housing with a lid hingedly connected to the housing. The lid has an open position and a closed position. In a preferred embodiment, a heating element is connected to the lid. A removable container, which is divided into a first compartment and a second compartment when the lid is closed, is operatively arranged to fit in the housing. Heat emitted from the heating element increases the temperature of the contents of the first compartment while the contents of the second compartment remain at the approximate temperature prior to activation of the heating element.

In some embodiments, the present disclosure provides an apparatus that includes a housing with a lid having a heating component connected, preferably attached, to the lid. The lid is hingedly connected to the housing. The lid has an open position and a closed position. A removable container operatively arranged to fit in the housing has a first compartment and a second compartment. The container is suited to hold a liquid and the heating component emits heat into the first compartment when the heating element is activated.

In some aspects, the apparatus further comprises an insulating barrier that is disposed in the container when the lid is in a closed position. In the closed position, the insulating barrier is adjacent the heating element and forms a first compartment on a first side of the insulating barrier and second compartment on a second side of the insulating barrier that is opposite the first side. The apparatus can have the insulating barrier connected to the lid so that the insulating barrier is adjacent the heating element, and the heating element is perpendicularly connected to the lid.

In some embodiments, the heating element is disposed at another location on the apparatus other than connected to the lid. A wall connected to the lid can still function to separate the removable container into a first compartment and a second compartment when the lid is closed. An insulating barrier can be included on the wall.

In some aspects, the heating element partitions the first compartment from the second compartment when the lid is in a closed position, and any contents in the first compartment are sealed from entering the second compartment.

Some embodiments of the apparatus include a spraying system that sprays a liquid from the second compartment into the first compartment that has been heated after a cleaning cycle has completed.

In some embodiments, the first compartment is pivotally connected to the second compartment. The first compartment tilts to mix a liquid in the first compartment with a liquid in the second compartment, after the heating element is deactivated. Removal of the heating element can facilitate the tilting action required to mix the contents.

In some embodiments, the apparatus has a heating element, a first compartment for holding a liquid heated by the heating element, and a second compartment for holding a cooler liquid. The first compartment can submerge into the second compartment so that cooler liquid of the second compartment can mix with hot liquid of the first compartment to cool the liquid of the first compartment, after the heating element is deactivated.

In some embodiments, the first compartment is on a spring and floating ball valve device. After the first compartment has been heated and a cleaning cycle has completed, it is compressed and submerged into the cooler liquid contained in the second compartment. Release of the compression removes the first compartment from the second compartment.

Some embodiments of the apparatus include three compartments, namely a first compartment for holding heated liquid and placement of the dentures therein, a second compartment holding cool liquid and operatively connected to the first compartment, and a third compartment initially empty but adapted to move into the second compartment. Suitably, the empty third compartment is on a spring device and when compressed causes the cooler liquid in the second compartment to flow into the first compartment. This causes the heated liquid in the first compartment to flow into the third compartment. Release of the compression removes the third compartment from the second compartment.

In some embodiments, the apparatus comprises a dispensing mechanism connected to the lid in which a removable cartridge with a plurality of tablets enclosed therein can be inserted into the dispensing mechanism. The dispensing mechanism is operatively arranged to eject the tablets from the cartridge. The cartridge can be disc shaped, and the tablets can be arranged at a perimeter of the cartridge. Also, the dispensing mechanism is arranged to rotate the cartridge.

The above-described and other features and advantages of the present disclosure will be appreciated and understood by those skilled in the art from the following detailed description, drawings, and appended claims.

### BRIEF DESCRIPTION OF THE SEVERAL VIEWS OF THE DRAWINGS

FIG. 1 is a perspective view of a dental appliance cleaning apparatus of the present disclosure.

FIG. 2 is a perspective view of the dental appliance cleaning apparatus of FIG. 1 in an open position.

FIG. 3 is a perspective view of the dental appliance cleaning apparatus of FIG. 1 with the container removed.

FIG. 4 is a cross-sectional view of the dental appliance cleaning apparatus shown in FIG. 1, taken generally along line 4-4 in FIG. 1.

FIG. 5 is a cross-sectional view of the dental appliance cleaning apparatus shown in FIG. 1, taken generally along line 5-5 in FIG. 1.

FIG. 6 is a cross-sectional view of the dental appliance cleaning apparatus shown in FIG. 1, taken generally along line 6-6 in FIG. 1.

FIG. 7 is a perspective view of a dispensing mechanism separated from the cleaning apparatus shown in FIG. 1.

FIG. 8 is an alternative embodiment of a container for the cleaning apparatus shown in FIG. 1

FIG. 9 is an alternative embodiment of the dental appliance cleaning apparatus of the present disclosure.

FIG. 10 is a cross-sectional view of the dental appliance cleaning apparatus shown in FIG. 9, taken generally along line 10-10 shown in FIG. 9.

FIG. 11 is a perspective view of an alternative embodiment of the dental appliance cleaning apparatus of the present disclosure.

FIG. 12 is a cross-sectional view of the dental appliance cleaning apparatus shown in FIG. 11, taken generally along line 12-12 in FIG. 11.

FIG. 13 is a perspective view of an alternative embodiment of the dental appliance cleaning apparatus of the present disclosure.

FIG. 14 is a cross-sectional view of the dental appliance cleaning apparatus shown in FIG. 13, taken generally along line 14-14 in FIG. 13.

FIG. 15 is a perspective view of an alternative embodiment of the dental appliance cleaning apparatus of the present disclosure.

FIG. 16 is a cross-sectional view of the dental appliance cleaning apparatus shown in FIG. 15, taken generally along line 16-16 in FIG. 15.

### DETAILED DESCRIPTION OF THE INVENTION

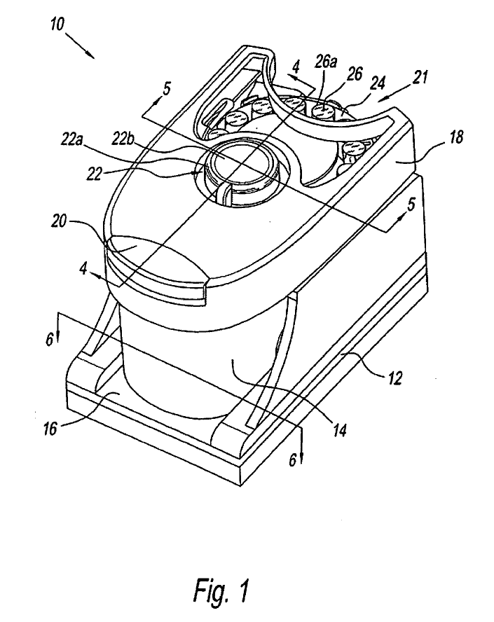

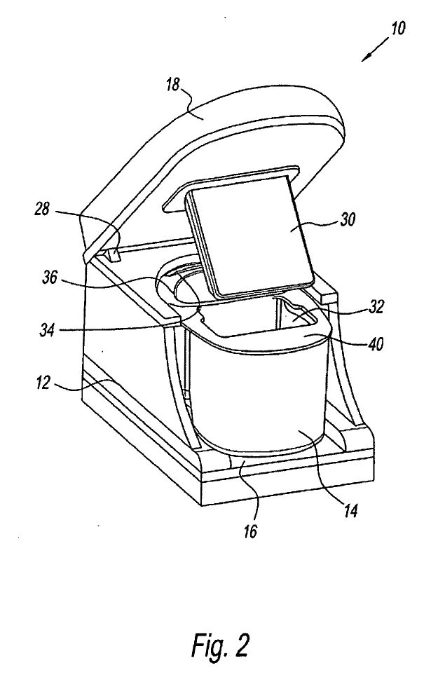

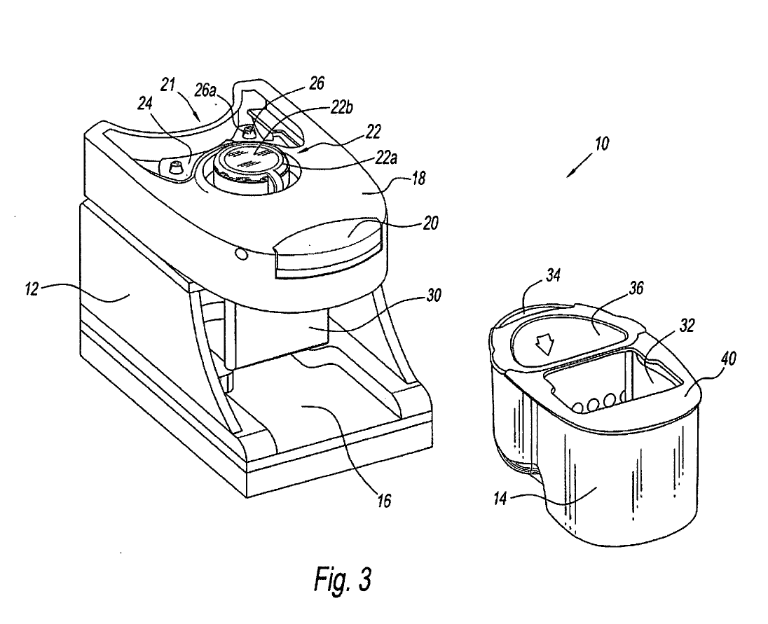

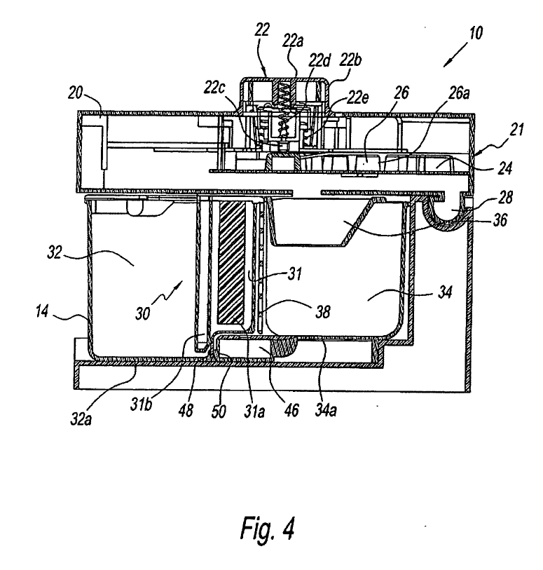

Referring to the drawings and in particular to FIG. 1, cleaning apparatus is shown generally represented by reference numeral 10. The apparatus 10 has a housing 12, a container 14, a lid 18 with a wall 30 (shown in FIG. 2), preferably having a heated element 31 (shown in FIG. 4), and a cartridge 24 that fits inside lid 18 through opening 21. Cartridge 24 has cleaning tablets 26. Container 14 is a container designed to hold liquid in separate compartments 32 and 34 (shown in FIG. 3) as the heating element 31 heats the contents of one compartment. Container 14 is a watertight receptacle that is shaped so that it may be separated into two compartments. Container 14 provides the receptacle that holds items, such as dental appliances, during a heating/cleaning cycle.

After a heating cycle/cleaning cycle is completed, wall 30 with heating element 31 is removed and the contents of the unheated compartment mix with the contents of the heated compartment, thereby almost immediately cooling the heated compartment. Thus, this provides a user the opportunity to promptly remove dental appliances that have cleaned in apparatus 10. It should be understood that although heating element 31 is shown adjacent to wall 30, in some aspects, the heating element is disposed at another location on apparatus 10.

Thus, apparatus 10 provides a system with a cleaning cycle that cleans a dental appliance in a heated cleaning solution, which eliminates bacterial growth in pores and crevices, and then cools the cleaning solution to enable a user to remove the dental appliances seconds after the cleaning cycle is complete. The solution is any medium that contains antimicrobial or an agent that provides active oxygen and also provides sufficient conductivity to increase temperature to a desired range. Such antimicrobial agents include, but are not limited to, active chlorine compounds (e.g. chlorine dioxide, hypochlorites); alcohols, including aliphatic alcohols (e.g. ethanol) or aromatic alcohols (e.g. phenoxyethanol, phenoxypropanol, benzyl alcohol); quaternary ammonium compounds (e.g. benzalkonium chloride, benzethonium chloride) and other amine compounds (e.g. chlorhexidine salts); weak organic acids such as benzoic acid, salicylic acid, sorbic acid, lactic acid, hydroxybenzoic acid and derivatives (e.g. parabens); thymol, menthol, eucalyptol and other plant extracts and oils; zinc salts, and combinations thereof.

Apparatus 10 provides a system and method of cleaning dental appliances, including, but not limited to dentures, bridges, orthodontic retainers and appliances, protective mouthguards, and nightguards to prevent bruxism and/or temporomandibular joint (TMJ) syndrome. This disclosure provides the understanding that dental appliances have pores and crevices, which traditional cleaning methods and devices are ineffective at cleaning. As noted, pores that are inadequately cleaned can harbor bacteria that can emit unpleasant odors. To eliminate the bacterial growth in these pores, and in order to properly clean dental appliances by removing any substances that lodge inside pores or grooves, this disclosure describes a cleaning method and apparatus that addresses the deficiencies of present cleaning methods and apparatuses.

Housing 12 provides an area for container 14 that the container rests in. Housing 12 and container 14 may be constructed of opaque or transparent plastic or various other substances. Housing 12 has a groove 16 that complements container 14. The complementary shape of groove 16 enables housing 12 to retain container 14. The complementary shape of groove 16 may also provide a guide for the removal and placement of container 14 in housing 12.

Housing 12 has pivotally connected thereto lid 18. Lid 18 may be constructed of opaque or transparent plastic or various other substances. Pivoting lid 18 lifts upward to open apparatus 10 that enables container 14 to be removed from housing 12. Positioned proximate one end of apparatus 10 on lid 18 is release mechanism 20 that may be pressed, or actuated in another manner, to release lid 18 from a closed position.

In some embodiments, a dispensing mechanism 22 is positioned on top of lid 18. Dispensing mechanism 22 may be positioned at various other locations on apparatus 10, such as on the side, front or back of apparatus 10, or the side of lid 18. Dispensing mechanism 22 has a dial 22a that rotates along a radial path that is on roughly the same plane as the top surface of lid 18. In some aspects, dispensing mechanism 22 also has a button 22b that moves along a vertical path that is perpendicular to the radial path of dial 22a. In some aspects, dispensing mechanism 22 combines the features of dial 22a and button 22b in a single dispensing mechanism 22. Thus, dispensing mechanism 22 may be a single component that rotates and depresses to dispense tablets 26.

In some aspects, cartridge 24 is a disc-shaped blister package that has individual tablets 26 contained in small blister cavity 26a that are uniformly dispersed proximate the outer periphery of cartridge 24. Blister cavities 26a in cartridge 24 are approximately the size of tablets 26. In some aspects, cartridge 24 includes an additional blister cavity (not shown) that contains a breath freshening or flavored substance that is dispensed by actuating dispensing mechanism 22.

Cartridge 24 is loaded into apparatus 10 by inserting the cartridge in an opening 21 in lid 18. Opening 21 is sufficient to receive cartridge 24 into lid 18 to enable dispensing mechanism 22 to engage the cartridge. In some embodiments, improperly loaded cartridge 24 is correctly positioned by radially advancing dial 22a or dispensing mechanism 22. A loaded cartridge 24 that is not in the correct radial position is aligned properly by advancing dial 22a or dispensing mechanism 22 until proper alignment is achieved.

Button 22b may be depressed or manipulated in some other manner to release tablets 26 that are contained in cartridge 24. Pressure exerted on the top of cartridge 24 forces tablet 26 from the cartridge, releasing tablet 26 to fall inside container 14.

Referring to FIG. 2, lid 18, which is shown in the open position, is pivotally connected to housing 12 by pivot joint 28. Pivot joint 28 enables lid 18 to open sufficiently so that container 14 can be placed in apparatus 10, or be removed from the apparatus. Lid 18 is in its open position when the lid is raised or pivoted away from housing 12 to enable container 14 to be removed from the housing. Lid 18 is in its closed position when the lid is lowered or pivoted toward housing 12 until it rests atop the housing.

Lid 18 can be connected to housing 12 using another attachment method. For example, lid 18 can be detachably connected (not shown) to housing 12. In such an arrangement, lid 18 is completely removable from housing 12 when the lid is in its open position, and the lid is connected to the housing when the lid is in its closed position.

In some aspects, wall 30 has heating element 31 (not shown). Wall 30 is a linear part that is connected perpendicularly to the underside of lid 18. Wall 30 can be a component with a heating element 31 that emits heat when activated. Wall 30 and heating element 31 are shown as a thin rectangular tongue that is positioned transverse to lid 18. However, wall 30 and heating element 31 may be of various shapes and sizes other than the preferred embodiment shown and described herein. Such other shapes and sizes include, but are not limited to, a slender tube or a round-edged heating element. Also, heating element 31 may be positioned longitudinal to lid 18, as opposed to the preferred transverse position. In addition, heating element 31 may be positioned at various other positions (not shown) in apparatus 10, other than adjacent wall 30, which are proximate compartment 32.

In order for container 14 to be removed from apparatus 10, lid 18 must be raised to a height where wall 30 and heat element 31 are no longer obstructing container 14 from sliding in groove 16 out of housing 12. Container 14 may be removed from groove 16 of housing 12 when lid 18 is in an open position.

Referring to FIG. 3, container 14 is removed, and lid 18 is shown in the down or closed position. Container 14 has two compartments 32 and 34. Compartment 34 is designed to hold dental appliances placed therein. Both compartments 32 and 34 are capable of holding a liquid, such as water. A liquid placed into one compartment, such as 34, distributes between compartments 32 and 34. Compartments 32 or 34 may also include a funnel 36 that directs liquid or cleaning tablets into either compartment. Funnel 36 may also serve to hold dental appliances in position during a cleaning cycle.

In some aspects, a strainer 38, shown in FIG. 4, separates compartments 32 and 34. Strainer 38 is a fenestrated wall that prevents dental appliances placed in compartment 34 from migrating into compartment 32. Small apertures in wall 38 enable liquid and small particles to pass between compartment 34 and compartment 32, but prevent larger items, such as dental appliances, from migrating from compartment 34 to compartment 32. Compartment 34 is intended as the cleaning compartment that holds dental appliances during and subsequent a cleaning cycle.

Referring to FIGS. 3 through 6, container 14 accepts wall 30 with heating element 31 when lid 18 is in a closed position. By accepting heating element 31, liquid inside compartment 34 may be heated when the heating element is activated. Insertion of wall 30 into container 14 serves to partition container 14 into two separate compartments, i.e., compartments 32 and 34. Fully inserting wall 30 into container 14 enables the wall to act as a partition between compartments 32 and 34 so that the contents in compartment 34 are separated from the contents in compartment 32.

Apparatus 10 is used to perform cleaning cycles in which items placed in compartment 34 are subsequently exposed to a heated aqueous solution that may contain a cleaning agent. A cleaning cycle may commence after container 14 is fully inserted in housing 12, lid 18 is closed, and wall 30 with heating element 31 is inserted into container 14. The cleaning cycle involves heating the contents of compartment 34 to an optimal cleaning temperature by activating heating element 31, and actuating dispensing mechanism 22 to release a tablet 26 into container 14.

As shown, wall 30 separates compartment 34 from compartment 32 when lid 18 is in the closed position. Container 14 provides the receptacle that holds dental appliances during a cleaning cycle. Heat generated by heating element 31 of wall 30 raises the temperature of the liquid inside compartment 34. The optimal temperature of the liquid inside compartment 34 is 65 degrees Celsius, but temperatures that range from 55 to 70 degrees Celsius are satisfactory. Temperatures set forth in this application may vary +/− one degree Celsius.

The optimal temperature range is intended to accentuate the cleaning effectiveness of any cleaning formula or tablet used to clean the dental appliance placed in apparatus 10. Depending on the cleaning formula or tablet used and the item that is to be cleaned, the optimal temperature ranges from 50 to 70 degrees Celsius. Thus, heating element 30 may be calibrated to reach various temperature ranges from 50 to 70 degrees Celsius, and preferably 60 to 65 degrees Celsius.

Heat generator 31a produces the heat emitted from heating element 31, and heat generator 31a is housed inside heating element 31. Heat generator 31a may be various shapes including a tubular element that is u-shaped, round, coiled, or rectangular, contained in a stainless steel, aluminum or other metal housing, or a PTC element that is round, rectangular, square, ring or donut style, contained in a stainless steel or aluminum housing. Heat generator 31a is positioned proximate compartment 34 when lid 18 is in the closed position. Heat emitted from heat generator 31a is conducted into the liquid inside compartment 34 until the temperature of the liquid reaches the optimal temperature that the heat generator has been calibrated to reach. In some embodiments, heat generator 31a has a regulator mechanism that senses change in the temperature of the liquid in compartment 34 and adjusts the heat output from the heat generator to maintain the temperature approximately near the optimal temperature or temperature range. As the temperature in compartment 34 raises or lowers, heat generator 31a is adjusted, that is activated or deactivated, in order to maintain the optimal temperature of the liquid.

In some aspects, a mixing system 46 is placed in apparatus 10, preferably in the base thereof, to achieve a uniform distribution of the heat generated by heat generator 31a. Mixing system 46 may implement a mixing wheel driven by a small motor (not shown) that spins magnets (not shown) in the mixing wheel or by ultrasonics or vibration. Relatively equal distribution of heated liquid in compartment 34 enables more effective and safe cleaning of the dental appliance placed therein and may reduce the occurrence of hot spots in compartment 34.

In some aspects, an ultrasonic cleaning device is used to assist in the removal of surface plaque on dental appliances. A transducer for ultrasonication is placed in apparatus 10, preferably adjacent to heating element 31. Ultrasonic cleaning device may also assist in raising the temperature of the liquid one to five degrees Celsius.

Heat provided by heat generator 31a is mainly confined to heating liquid or contents in compartment 34. Heat from heating element 31 and the heated liquid are confined to compartment 34 due to seal 48 and insulated barrier 31b. Insulated barrier 31b is either a layer of insulating material or a barrier or pocket that provides a sufficient insulating effect to suppress the conduction of heat to compartment 32. Insulated barrier 31b in wall 30 also provides a barrier that insulates heat from the heated liquid in compartment 34, and the heat generator 31a, from increasing the temperature of the liquid in compartment 32.

In some embodiments, heat may be provided by a chemical heating due to an exothermic reaction. This may be provided by a combination of chemicals in tablet 26 or a separate tablet. Suitable compounds for creating an exothermic reaction include calcium chloride, calcium hydroxide and calcium oxide, separately, or in combination with magnesium acetate, magnesium hydroxide, magnesium oxide, magnesium sulfate, sodium acetate and sodium chloride. A combination of calcium oxide and a water soluble compound is preferred.

In some embodiments, seal 48 is disposed around lip 50, which is disposed on a single plane around the walls and floor of container 14 between compartments 32 and 34. As lid 18 is closed and wall 30 is positioned between compartments 32 and 34, wall 30 abuts seal 48 and serves to seal compartment 34 from compartment 32. In some aspects, seal 48 is formed from an elastomeric material, such as rubber, and is compressed as wall 30 is moved against seal 48. The seal formed between wall 30 and seal 48 provides that heated liquid will generally be contained inside compartment 34.

In some aspects, seal 48 is disposed on wall 30 and seals compartment 34 from compartment 32 when wall 30 is lowered with lid 18. A planar surface of wall 30, which has seal 48 connected thereto, abuts lip 50 to seal compartment 34 from compartment 32.

Isolation of heated liquid to compartment 34 enables liquid held within compartment 32 to remain approximately the temperature of the liquid that is initially placed inside container 14. That is, if cool or cold liquid is placed in container 14, the liquid remains approximately at that same temperature in compartment 32, i.e., cool or cold, even after submerged heating element 31 heats the liquid in compartment 34. Thus, separation of compartment 34 from compartment 32 enables container 14 to have a heated liquid side and a cool liquid side when lid 18 is closed and the heating element 31 is activated.

Cleaning cycles may commence each time lid 18 is lowered into a closed position and wall with heating element 31 is submerged into container 14, and then activated. Partitioning container 14 into compartments 32 and 34 with the submersion of heating element 31 prepares apparatus 10 to commence a cleaning cycle. These separate compartments in container 14 enable cleaning cycles to occur in a single compartment. For example, cleaning cycles involve heating of compartment 34 to an optimal temperature, as described above, for an optimal cleaning duration. Depending on the liquid used in container 14, the temperature chosen, the composition of cleaning tablets 26, and item being cleaned the optimal cleaning duration may range from 3 minutes to 15 minutes, but a period outside of this range is also considered within the scope of this disclosure.

In some aspects, dispensing mechanism 22, dial 22a, and/or button 22b serve as the switch that activates heating element 30. For example, depression of button 22b commences a cleaning cycle by activating heat generator 31a. Liquid in compartment 34 begins to heat until reaching an optimal temperature and continues to maintain that temperature until the optimal cleaning duration has been reached. A safety device (not shown) may be included in dispensing mechanism 22 to prevent the activation of heating element 31 if a tablet is not ejected after the depression of dispensing mechanism 22 and/or button 22b.

Dental appliances placed within compartment 34 remain in the heated contents until the cleaning cycle is completed. After the cleaning cycle ends, heat generator 31a is turned off, either by a manual switch or an automatic system (not shown). In some aspects, an internal governor (not shown) controls a timing mechanism that ends a heating cycle. An indicator may be incorporated into apparatus 10 that senses or enables a user to sense when a cleaning cycle is complete, such as after a set cleaning period has elapsed. A light or audible alert may be used to signal the end of a cleaning cycle.

Upon completion of a cleaning cycle, lid 18 may be opened and pivoted into the upper position. As lid 18 is pivoted upward, wall 30 emerges from container 14 and the liquid from separate compartments 32 and 34 are no longer partitioned from each other. Removal of wall 30 enables the cool liquid in compartment 32, which has been insulated from heating element 31 and the heated liquid in compartment 34, to mix. Mixing system 46 may further facilitate the mixing of the liquid from compartments 32 and 34.

As the cooler liquid of compartment 32 enters compartment 34, and vice versa, the overall temperature of the liquid in container 14 approaches a uniform temperature that is cooler than the temperature that the heated liquid in compartment 34 reached. By mixing of the liquid from compartments 32 and 34, the temperature of the mixed liquid becomes more tolerable to touch, and alleviates the risk of injury that would occur if the heated liquid from compartment 34 contacts skin. However, after the liquids in compartments 32 and 34 are mixed, the dental appliances may be removed by hand about 5 second to about 30 seconds after apparatus 10 is opened. Also, strainer 38, lying between compartments 32 and 34, prevents the migration of dental appliances from compartment 34 while the liquid in the compartments 32 and 34 mixes.

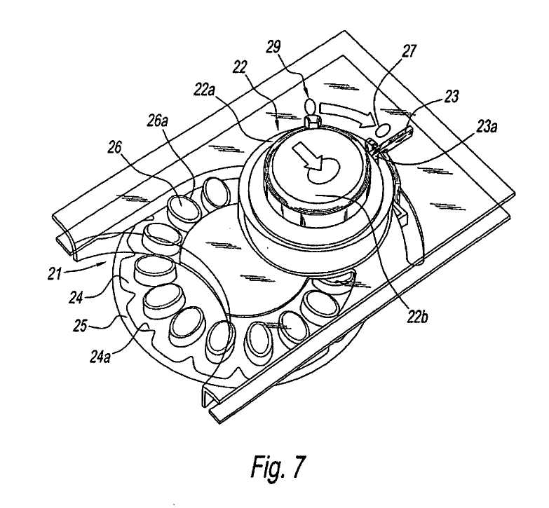

As noted above, dispensing mechanism 22 is positioned near the center of apparatus 10 on lid 18 and is structured to release tablets 26 from cartridge 24. FIG. 7 shows one representation of dispensing mechanism 22. In this representation, dispensing mechanism 22 is rotatably connected to lid 18 and has regions that interact with cartridge 24. Dial 22a and/or dispensing mechanism 22 is linked to a prong 23 that enables the rotation of cartridge 24 as dial 22a and/or dispensing mechanism 22 is rotated. Cartridge 24 is inserted into opening 21 in lid 18 and the cartridge is set on top cartridge holder 25. Notches 24a in cartridge 24 may interact with tabs on cartridge holder 25. Notches 24a can be of any shape provided that the notches can be gripped for movement, however a v-shape notch is preferred. Prong 23 is connected to dispensing mechanism 22 and/or dial 22a, and prong 23 interacts with a slot 23a on cartridge holder 25 that enables the rotation of dial 22a or dispensing mechanism 22 to cause the rotation of cartridge holder 25 and thereby the rotation of cartridge 24. For example, as dispensing mechanism 22 and/or dial 22a is rotated, prong 23 is rotated, which causes the rotation of cartridge holder 25 and cartridge 24.

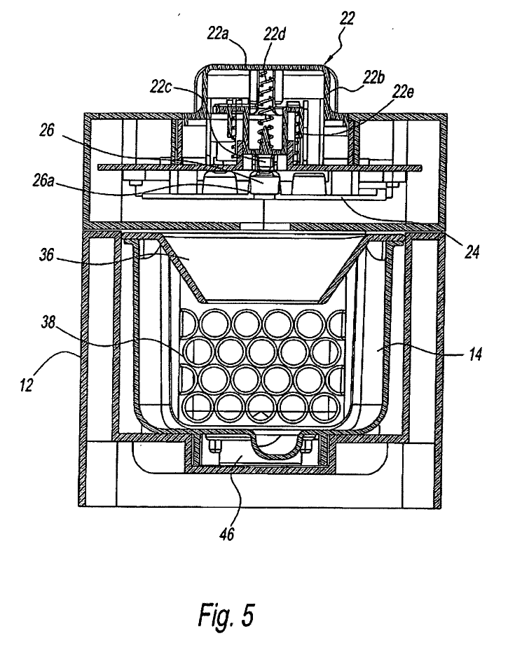

In some aspects, dial 22a is arranged to rotate clockwise roughly 45 degrees to advance cartridge 24 to an ejection position 27 and dial 22a has a neutral position 29 that dial 22a returns to after being rotated to advance cartridge 24. In this arrangement, a spring system 22d shown in FIG. 5, engages dial 22a to return dial 22a back to the neutral position after rotating 45 degrees.

Tabs 25a engage cartridge notches 24a and as dial 22a is rotated, cartridge 24 rotates. Dispensing mechanism 22 is positioned to center tablets 26, which are held inside blister cavity 26a on cartridge 24, under post 22c as the cartridge is rotated. Cartridge 24 is rotated after each cleaning cycle to rotate a new tablet 26 into position under post 22c for a subsequent cleaning cycle.

Referring to FIG. 5, button 22b is capable of being depressed to send post 22c down toward tablet 26 that is contained inside a blister cavity 26a in cartridge 24. Button 22b is engaged with post 22c in such a way that depression of button 22b causes post 22c to move downward against cartridge 24. Post 22c continues to press against blister cavity 26a in cartridge 24, containing tablet 26, as button 22b is depressed until the tablet is eventually ejected. Ejection of tablet 26 from cartridge 24 is more precisely accomplished by a deliberate downward thrust on dispensing mechanism 22 and/or button 22a. Preferably, the downward thrust is swift. A thin sheet of material at the back of cartridge 24, opposite the contact point for post 22c, gives way to pressure exerted on tablet 26 to release the tablet. The packaging that holds tablet 26 in cartridge 24 may be blister packaging, or various other similar type package. The positioning of cartridge 24 and dispensing mechanism 22 over compartment 34 enables tablet 26 to be ejected into compartment 34 upon the depression of button 22b and post 22c.

Spring 22d and 22e provide resistance that returns button 22b and post 22c, respectively, to the original position that they existed in prior to being depressed.

As noted above, commencement of a heating cycle, and release of a tablet 26 are initial steps that begin a cleaning cycle. Tablet 26 may have various formulations and components. The components generally include substances that may effectively clean various dental appliances. Heated liquid in compartment 34, which receives tablet 26, may serve to better dissolve tablet 26 and improve the cleaning effectiveness of chemical agents in the tablet. Heat has been shown by the present disclosure to be a major factor that improves the cleaning effect of dental appliance cleaning formulations, and enables the deep cleaning needed to eliminate buildup on surfaces and inside pores and grooves found in many dental appliances. Particularly, this is achieved by the heated liquid that is maintained at the optimal cleaning temperature and/or range for a particular duration.

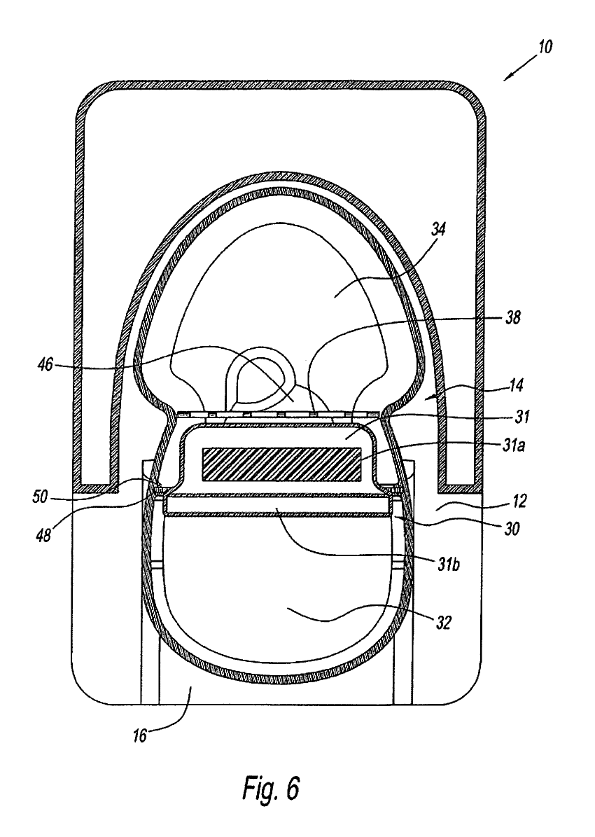

Referring to FIG. 6, wall 30 is shown in the closed position. In this position, the edge of wall 30 abuts seal 48 held in place by lip 50. Other embodiments of apparatus 10 use alternative shaped heating elements, or eliminate seal 48, but in embodiments utilizing seal 48 the interaction of the edge of wall 30 with the seal, as shown, precludes the mixing of liquid in compartments 32 and 34 when lid 18 and wall 30 are in the closed position.

An alternative embodiment of a container for the cleaning apparatus is shown in FIG. 8. In this alternative arrangement, container 60 replaces container 14 of the FIG. 1 embodiment. Container 60 has a first compartment 62 and a second compartment 64. First compartment 62 is pivotally connected to second compartment 64, preferably to the interior walls of the second compartment. Posts 66 are positioned proximate one end of compartment 62. Notches 68 are positioned proximate one end of compartment 64. Posts 66 are complementary to notches 68 so that posts 66 are free to pivot within notches 68. Posts 66 can be either connected or integrally attached to the exterior of compartment 62.

It is preferred that posts 66 of compartment 62 rest within notches 68 so that compartment 62 can be removed from compartment 64. Thus, compartment 62 is a separate container that rests within a slightly larger compartment 64.

In some aspects, the resting position of first compartment 62 is tilted slightly toward one end of container 60. Compartment 62 is contoured to receive dental appliances and when compartment 62 is tilted dental appliances will remain inside that compartment. In a tilted position, any liquid in compartments 62 and 64 would mix. However, if pressure is exerted on compartment 62 proximate posts 66, compartment 62 is shifted from a tilted position to a roughly level position. In this level position, compartment 62 can retain the liquid contents of compartment 62 separate from compartment 64. Therefore, it is possible to have separate contents inside the compartments 62 and 64.

Compartment 62 can be heated with a heating element, similarly to the way compartment 32 is heated, and second compartment 64 remains unheated during a heating cycle. In alternative embodiments, compartment 64 is the compartment that is heated and dental appliances are placed within compartment 64 for cleaning. Container 60 can be used with apparatus 10. Wall 30, when inserted in container 60, supports compartment 62 in a level position when lid 18 is fully closed. In some aspects, wall 30 will require an alternative shape than depicted in the figures of the present disclosure in order to fit properly inside container 60 and properly pivot compartment 62 into a level position.

To initiate a cleaning cycle of apparatus 10 with container 60 replacing container 14, first container 60 is filled with cool liquid to an appropriate level so that both compartments 62 and 64 will have a sufficient liquid level. After inserting compartment 60 into apparatus 10, lid 18 is closed so that wall 30 tilts compartment 62, toward a level position, and when lid 18 is in a fully closed position, compartment 62 is in its level position. Heating element 31 can be activated by various means after lid 18 is closed. Since compartment 62 is now in a level position, the contents of compartment 62 are segregated from the contents of compartment 64. Thus, any substances, heat, or other physical or chemical changes that are subjected on the contents of compartment 62 are isolated from compartment 64. Therefore, if a cleaning tablet is added to compartment 62 and/or the contents of compartment 62 are heated, compartment 64 remains unaffected and remains in the original state.

Dispensing mechanism 21 and wall 30 function with container 60 just as those components function with container 14.

Following a cleaning cycle and deactivation of heating element 31, wall 30 and the heating element is removed and as lid 18 is pivot upward, first compartment 62 pivots to empty, or at least partially empty, the liquid inside that compartment into second compartment 64. Pivoting of first compartment 62 enables the cool liquid in second compartment 64 to mix with the heated liquid and contents of first compartment 62, and the mixing causes the cooling of the heated contents of first compartment 62.

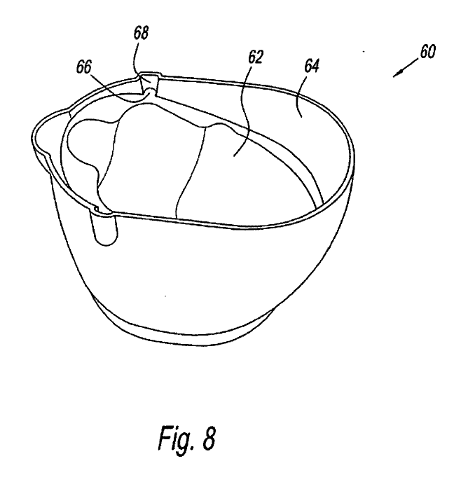

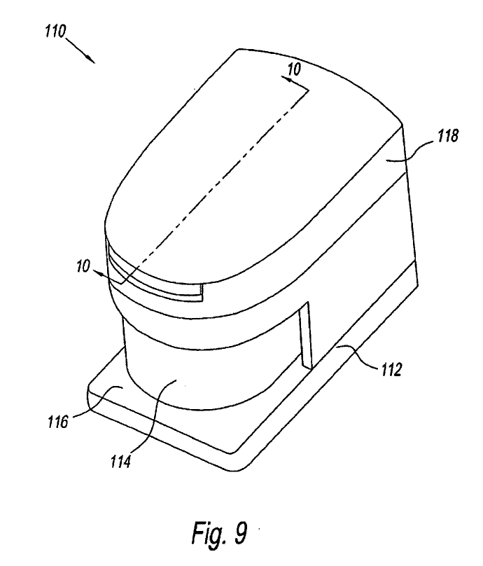

FIG. 9 is an alternative embodiment of the dental appliance cleaning apparatus of the present disclosure. In this embodiment, apparatus 110 has a housing 112 with a container 114 that fits within a slot 116 in the housing. Lid 118 is pivotally connected to housing 112 to enable access to the interior of apparatus 110.

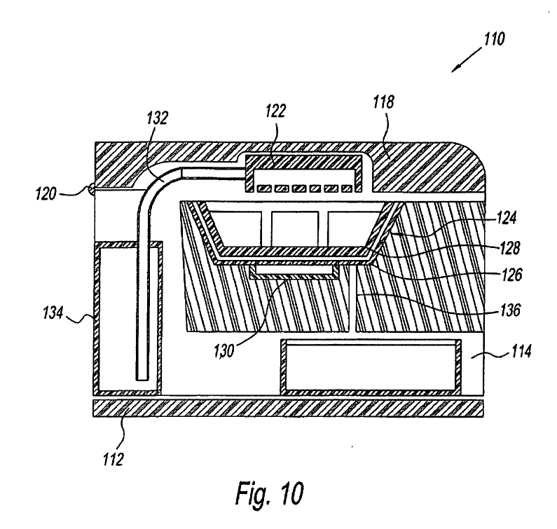

Referring to FIG. 10, lid 118 is pivotally connected to housing 112 with hinge 120. This connection enables lid 118 to be lifted upward to expose spray mechanism 122 and cleaning compartment 124. Apparatus 110 has a container 126 that is suitable to hold a liquid, and a basket 128 that can hold items to be cleaned, such as dental appliances. Beneath container 126 is a heating element 130 that is calibrated to maintain the temperature of liquid held in container 126 at an optimal temperature for cleaning the items contained within the container. Heating element 130 can implement various heating components known in the art that are capable of maintaining the proper temperature for a sufficient time period. Heating element 130 can commence a heating cycle after lid 118 is closed and terminate a heating cycle after a set cleaning time period has transpired.

After the heating cycle has completed and heating element 130 has been deactivated, the contents of container 126 are drained into receptacle 114. In some aspects, spraying mechanism 122 utilizes a pumping component that draws liquid through tube 132 to spraying head 122. A second container 134 can hold an unheated or cooled liquid that supplies liquid to tube 132 and spraying mechanism 122. Liquid from container 134 is sprayed by spraying mechanism 122 on the contents of container 126 that have that has just undergone a cleaning cycle. The sprayed liquid provides cooling that enables the items in container 126 to be handled moments after the cleaning cycle has ended.

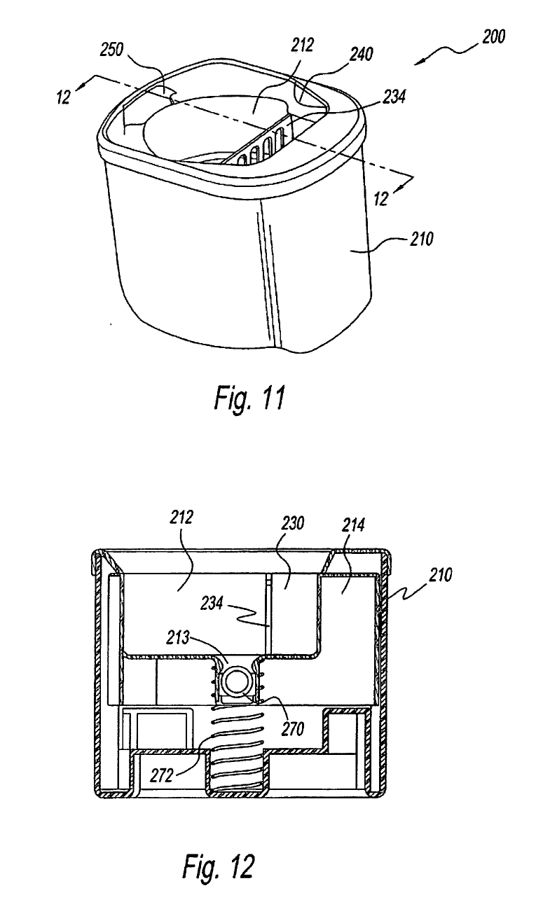

FIGS. 11 and 12 show an alternative embodiment of the dental appliance cleaning apparatus of the present disclosure. In this embodiment, apparatus 200 has a container 210 and a lid (not shown) with an opening. Container 210 has a first compartment 212 and a second compartment 214, both suitable to hold a liquid. Compartment 212 can hold dental appliances placed therein.

Compartment 212 can be heated with a heating element (not shown) placed in heating area 230. The heating element is calibrated to maintain the temperature of liquid held in compartment 212 at an optimal temperature for cleaning the dental appliances contained therein. Compartment 214 remains unheated during the heating cycle. A strainer 234 separates compartment 212 and heating area 230.

On each side of heating area 230, bubbler channels 240 are positioned. Bubbler channels 240 provide openings for the flow of liquid between compartments 212 and compartment 214. Compartments 212 and 214 may be filled with liquid prior to cleaning. Liquid in compartment 212, compartment 214 and bubbler channels 240 are removed by pouring spout 250. The base of compartment 212 has an opening 213 leading into compartment 214. When liquid is present in compartment 212 and compartment 214, opening 213 remains sealed by the floating ball valve 270, which rests within the spring device 272.

After a heating cycle is completed and heating element within heating area 230 has been deactivated, a plunger (not shown) may be used to apply pressure to the base of compartment 212. As the plunger is moved downward, compartment 212 with the dental appliance therein is submerged into compartment 214. The floating ball valve 270 is displaced by the plunger so that opening 213 allows the heated liquid in compartment 212 and cool liquid in compartment 214 to mix. After the liquids mix, the liquid in compartment 212 becomes cooled. The release of the compression of spring 272 returns compartment 212 to the original position. This allows the dental appliances within compartment 212 to be handled moments after the cleaning cycle has ended.

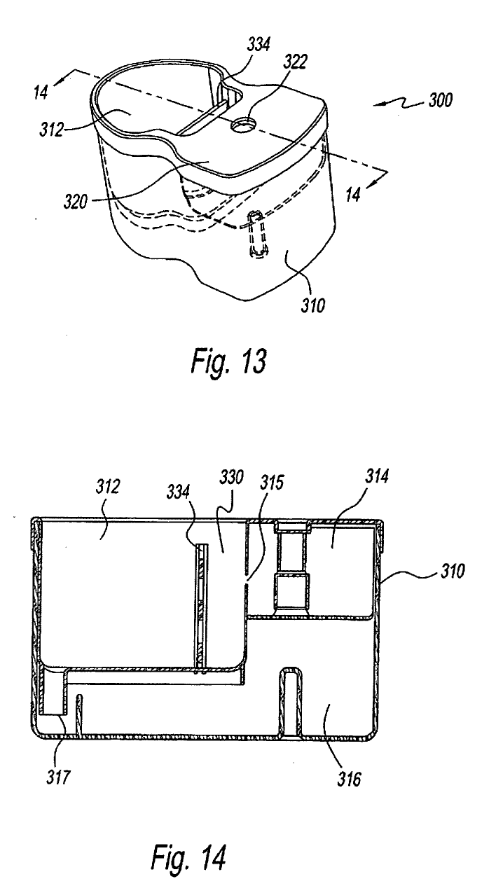

FIGS. 13 and 14 show an alternative embodiment of the dental appliance cleaning apparatus of the present disclosure. In this embodiment, apparatus 300 has container 310 and a lid 320 with an opening 322. Container 310 has a first compartment 312 and a second compartment 314, and a third compartment 316, all suitable to hold a liquid. Compartment 312 is designed to hold dental appliances places therein.

Compartment 312 can be heating with a heating element (not shown) placed in heating area 330. The heating element is calibrated to maintain the temperature of liquid held in compartment 312 at an optimal temperature for cleaning the dental appliance contained therein. Compartments 314 and 316 remain unheated during heating cycle. A strainer 334 separates compartment 312 and a heating area 330.

Compartment 314 rests on a spring device (not shown) connected to the base of compartment 316. Compartment 314 has an opening 315 to compartment 312. Compartment 312 has an opening 317 at its base to compartment 316. Compartments 312 and 316 are filled with liquid through compartment 312. Compartment 314 is not filled with liquid prior to cleaning cycle.

After a heating cycle is completed and heating element within heating area 330 has been deactivated, compartment 314 is lowered into compartment 316. Heated liquid from compartment 312 flows into compartment 314 through opening 315 and cooler liquid from compartment 316 enters compartment 312 through opening 317.

Once compartment 314 is released, it will return to its initial position due to a spring device (not shown) filled with the heated water from compartment 312. The cooler water in compartment 312 will then flow back through opening 317 into compartment 316 allowing the dental appliances in compartment 312 to dry.

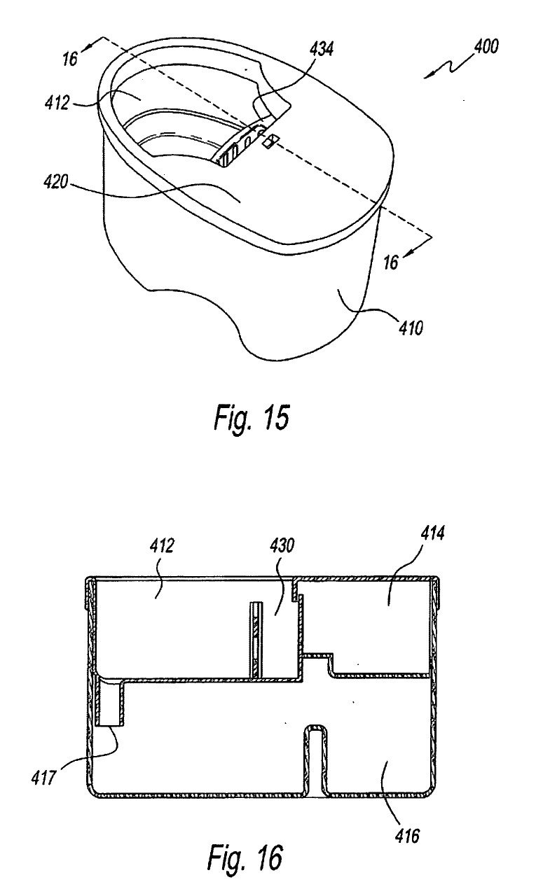

FIGS. 15 and 16 show an alternative embodiment of the dental appliance cleaning apparatus of the present disclosure. In this embodiment, apparatus 400 has container 410 and a lid 420. Container 410 has a first compartment 412 and a second compartment 414, and a third compartment 416, all suitable to hold a liquid. Compartment 412 is designed to hold dental appliances places therein.

Compartment 412 can be heating with a heating element (not shown) placed in heating area 430. The heating element is calibrated to maintain the temperature of liquid held in compartment 412 at an optimal temperature for cleaning the dental appliance contained therein. Compartments 414 and 416 remain unheated during heating cycle. A strainer 434 separates compartment 412 and a heating area 430.

Compartment 414 rests on a spring device (not shown) connected to the base of compartment 416. Compartment 412 has an opening 417 at its base to compartment 416. Compartments 412 and 416 are filled with liquid through compartment 412. Compartment 414 is not filled with liquid prior to cleaning cycle.

After a heating cycle is completed and heating element within heating area 430 has been deactivated, compartment 412 and compartment 414 are lowered into compartment 416. Heated liquid from compartment 412 flows into compartment 414 over the wall separating the compartments, while cooler liquid from compartment 416 enters compartment 412 through opening 417.

Once compartments 412 and 414 are released, they will return to their initial position due to a spring device. The cooler water in compartment 412 will then flow back through opening 417 into compartment 416 allowing the dental appliances in compartment 412 to dry.

Apparatuses 10, 60, 110, 200 and 300 are also capable of being used in applications other than cleaning dental appliances. Thus, this disclosure should not be interpreted to limit apparatuses 10, 60, 110, 200 and 300 to only that application. For instance, apparatuses 10, 60, 110, 200 and 300 are capable of being used to clean or sterilize other items such as, but not limited to, jewelry, tools, or coins. Other applications include using apparatuses 10, 60, 110, 200 and 300 as a water bath for conducting experiments that require a specific temperature for a specific time period. Reactants in the container may be maintained at the set temperature for a set period and then provide cooling once the set period has transpired.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

1. An apparatus, comprising:
a housing;
a lid hingedly connected to said housing, said lid having a wall positioned downward from said lid, wherein said lid has an open position and a closed position;
a heating element connected to said wall, wherein said heating element emits heat when said heating element is activated; and
a removable container suitable for placing inside said housing, wherein said wall is operatively arranged to divide said container into said first compartment and said second compartment when said lid is in said closed position.

2. The apparatus of claim 1, wherein said wall seals said first compartment from said second compartment when said lid is in said closed position.

3. The apparatus of claim 1, wherein said wall has an insulating member.

4. The apparatus of claim 3, wherein said insulating member inhibits heat emitted from said heating element from conducting into said second compartment.

5. The apparatus of claim 3, wherein said heating element is proximate said first compartment and said insulating member is proximate said second compartment when said lid is in said closed position.

6. The apparatus of claim 1, further comprising a seal disposed between said first compartment and said second compartment.

7-14. (canceled)

15. The apparatus of claim 1, wherein said container further comprises a hingedly connected flap.

16. The apparatus of claim 15, wherein said flap seals said first compartment from said second compartment when said lid is closed.

17. The apparatus of claim 1, wherein said container further comprises a strainer disposed between said first compartment and said second compartment.

18. The apparatus of claim 1, further comprising a warning system that signals when said heating element is activated.

19. The apparatus of claim 1, wherein said heating element further comprises a regulating mechanism that regulates within an optimal range a temperature of a liquid placed inside said container.

20. The apparatus of claim 19, wherein said optimal range is between 55 and 70 degrees Celsius.

21-23. (canceled)

24. An apparatus, comprising:
a housing;
a lid having a wall with a heating component fixedly connected thereto, wherein said lid is hingedly connected to said housing, and wherein said lid has an open position and a closed position; and
a removable container operatively arranged to fit inside said housing, wherein said container has a first compartment and a second compartment when said lid is in a closed position, wherein said container is suited to hold a liquid, wherein said heating component emits heat into said first compartment, but not in said second compartment, when said heating element is activated.

25-35. (canceled)

36. An apparatus, comprising:
an open container having a first compartment with a base and a second compartment positioned below said first compartment, the base having an opening for fluid connection to said second compartment, wherein first and second compartments can hold liquid;
a lid removably connected to said container to at least partially close said container, wherein said lid has an opening to receive a plunger;
a heating element placed in said first compartment, wherein said heating element emits heat when activated to heat the liquid in said first compartment, but not the liquid in said second compartment; and
wherein said first compartment is submerged into said second compartment to cool the liquid in said first compartment.

37-41. (canceled)

42. An apparatus, comprising:
an open container having a first compartment with a base, a second compartment positioned adjacent said first compartment and a third compartment positioned below said second compartment and adjacent and below said first compartment, the base having an opening for fluid connection to said third compartment, wherein said first compartment has an opening to said second compartment for fluid connection, wherein first and third compartments initially hold liquid therein;
a lid removably connected to said container to partially close said container, wherein said lid has an opening to said first compartment and an opening to said second compartment;
a heating element placed in said first compartment, wherein said heating element emits heat when activated to heat only the liquid in said first compartment;
wherein said second compartment is lowered into said third compartment so that heated liquid in said first compartment enters said second compartment and cooler liquid in said third compartment enters into said first compartment; and
wherein said second compartment is thereafter removed from said third compartment filled with heated liquid from said first compartment, and cooler liquid in said first compartment flows back into said third compartment, and said first compartment is empty of fluid.

43-44. (canceled)

45. An apparatus, comprising: an open container having a first compartment with a base, a second compartment positioned adjacent said first compartment and a third compartment positioned below said first compartment and said second compartment, the base having an opening for fluid connection to said third compartment, wherein said second compartment has a flap preventing fluid connection to said first compartment, wherein first and third compartments initially hold liquid therein;
a lid removably connected to said container to partially close said container, wherein said lid has an opening to said first compartment and said second compartment; and
a heating element placed in said first compartment, wherein said heating element emits heat when activated to heat only the liquid in said first compartment;
wherein said first compartment and said second compartments are lowered into said third compartment, wherein heated liquid in said first compartment enters said second compartment and cooler liquid in said third compartment enters into said first compartment; and
wherein said first compartment and said second compartment are thereafter removed from said third compartment, wherein said second compartment is filled with heated liquid from said first compartment, cooler liquid in said first compartment flows back into said third compartment, and said first compartment is empty of liquid.

46-47. (canceled)
